(19) United States
(12) Patent Application Publication
(10) Pub. No.: US 2016/0003857 A1
(43) Pub. Date: Jan. 7, 2016

(54) METHODS AND SYSTEMS FOR DETECTING POLYPHARMACY

(71) Applicant: Cecil Bennett, Conyers, GA (US)

(72) Inventor: Cecil Bennett, Conyers, GA (US)

(21) Appl. No.: 14/790,505

(22) Filed: Jul. 2, 2015

Related U.S. Application Data
(60) Provisional application No. 62/020,293, filed on Jul. 2, 2014.

(51) Int. Cl. G01N 33/94

(52) U.S. Cl. CPC G01N 33/94; G01N 2570/00; G01N 2560/00

(57) ABSTRACT

A method is disclosed for detecting polypharmacy in a subject that involves assaying a urine sample from the subject for a plurality of drugs or drug metabolites. The disclosed methods can further involve detecting potential adverse drug reactions in the polypharmacy of the subject. In addition, the method can further involve selecting a drug for treating the subject that does not interact with any drugs in the polypharmacy of the subject.

### CROSS-REFERENCE TO RELATED APPLICATIONS

This application claims benefit of U.S. Provisional Application No. 62/020,293, filed Jul. 2, 2014, which is hereby incorporated herein by reference in its entirety.

### BACKGROUND

Polypharmacy is a term that has been used in medical practice for decades. In traditional use, it means the use of multiple drugs in the treatment of a patient. However, over time, the potential for patient harm from polypharmacy has led to more specific definitions. One such definition is the prescription, administration or use of more medications than are clinically indicated or when a medical regimen includes at least one unnecessary medication. A simpler definition yet is the prescription of more medications than are clinically warranted. Several factors predispose patients to the risk of polypharmacy, including multiple care providers, an aging population (elderly patients are at greater risk for polypharmacy), complex drug therapies, multiple chronic conditions in a single patient and adverse drug reactions that may be interpreted as new medical conditions. Detecting and preventing polypharmacy, therefore, can help to reduce harmful drug interactions, remove ineffective therapies, reduce drug expenditures for patients, improve adherence and hopefully increase the patient's quality of life.

### SUMMARY

A method is disclosed for detecting polypharmacy in a subject that involves assaying a bodily fluid (e.g., a urine sample) obtained from the subject for a plurality of drugs or drug metabolites. The disclosed methods can further involve detecting potential adverse drug reactions in the polypharmacy of the subject. In addition, the method can further involve selecting a drug for treating the subject that does not interact with any drugs in the polypharmacy of the subject.

The details of one or more embodiments of the invention are set forth in the accompanying drawings and the description below. Other features, objects, and advantages of the invention will be apparent from the description and drawings, and from the claims.

### DETAILED DESCRIPTION

Disclosed is a method for detecting polypharmacy in a subject that involves assaying a bodily fluid (e.g., a urine sample) obtained from the subject for a plurality of drugs or drug metabolites.

In some embodiments, the plurality of drugs or drug metabolites includes 2, 3, 4, 5, 6, 7, 8, 9, 10 or more drug classes selected from the group consisting of antihypertensive drugs, anticoagulant drugs, cardiac glycosides, statins, herbal supplements, anti-diabetic drugs, thyroid hormone drugs, triptan drugs, appetite suppressants, antihistamine drugs, and over-the-counter (nonprescription) analgesic drugs. Non-limiting examples of antihypertensive drugs include ACE inhibitors, adrenergic receptor antagonists, alpha-2 adrenergic receptor agonists, dihydropyridine calcium channel blockers, non-dihydropyridine calcium channel blockers, beta blockers, and thiazide diuretics. Non-limiting examples of antihypertensive drugs include amlodipine, atenolol, clonidine, diltiazem, hydrochlorothiazide, lisinopril, metoprolol, propranolol, and verapamil. Non-limiting examples of anticoagulant drugs include clopidogrel and warfarin. Non-limiting examples of cardiac glycosides include digoxin. Non-limiting examples of statin drugs include atorvastatin, lovastatin, and simvastatin. Non-limiting examples of herbal include ginkgo, niacin, red yeast rice, and St. John's Wart. Non-limiting examples of anti-diabetic drugs include metformin. Non-limiting examples of thyroid hormone drugs include levothyroxine. Non-limiting examples of triptan drugs include almotriptan, sumatriptan, zolmitriptan, Non-limiting examples of appetite suppressant drugs include phentermine. Non-limiting examples of antihistamine drugs include pseudoephedrine. Non-limiting examples of over-the-counter (nonprescription) analgesic drugs include acetaminophen, ibuprofen, and salicylic acid.

In some embodiments, the plurality of drugs or drug metabolites includes one or more psychostimulant drugs or drug metabolites, such as a phenethylamine or a selective norepinephrine reuptake inhibitor. Non-limiting examples of phenethylamine drugs is selected from the group consisting of amphetamine, and methylphenidate. Non-limiting examples of norepinephrine reuptake inhibitor drugs comprises atomoxetine.

In some embodiments, the plurality of drugs or drug metabolites includes one or more anxiolytic drugs or drug metabolites, such as a benzodiazepine or an azapirone. Non-limiting examples of benzodiazepine drugs is selected from the group consisting of alprazolam, diazepam, and lorazepam. Non-limiting examples of azapirone drugs includes buspirone.

In some embodiments, the plurality of drugs or drug metabolites includes one or more opioid drugs or drug analytes. Non-limiting examples of opoid drugs include buprenorphine, codeine, morphine, oxycodone, hydrocodone, propoxyphene, and tramadol.

In some embodiments, the plurality of drugs or drug metabolites includes one or more prescription nonsteroidal anti-inflammatory drug (NSAID) or drug analytes. Non-limiting examples of prescription NSAID drugs include diclofenac and indomethacin.

In some embodiments, the plurality of drugs or drug metabolites includes one or more muscle relaxant drugs or drug analytes. Non-limiting examples of muscle relaxant drugs include baclofen, carisoprodol, and cyclobenzaprine.

In some embodiments, the plurality of drugs or drug metabolites includes one or more recreational drugs or drug analytes. Non-limiting examples of recreational drugs include cotinine (nicotine), ethyl glucuronide or ethyl sulfate (alcohol metabolites), and tetrahydrocannabinol (THC).

In some embodiments, the plurality of drugs or drug metabolites includes one or more antipsychotic drugs or drug analytes. Non-limiting examples of antipsychotic drugs include olanzapine and risperidone.

In some embodiments, the plurality of drugs or drug metabolites includes one or more anticonvulsant drugs or drug analytes. Non-limiting examples of neuropathic anticonvulsant drugs include carbamazepine, pregabalin, gabapentin, levetiracetam, and topiramate.

In some embodiments, the plurality of drugs or drug metabolites includes one or more acetylcholinesterase inhibitor drugs or drug analytes. Non-limiting examples of acetylcholinesterase inhibitor drugs include donepezil.

In some embodiments, the plurality of drugs or drug metabolites includes one or more nonbenzodiazepine hypnotic drugs or drug analytes. Non-limiting examples of hypnotic drug or sleep aids include eszopiclone, trazodone, zaleplon, and zolpidem.

In some embodiments, the plurality of drugs or drug metabolites includes one or more antidepressant drugs or drug analytes. Non-limiting examples of antidepressant drugs include amitriptyline, aripiprazole, bupropion, citalopram, duloxetine, escitalopram, fluoxetine, paroxetine, sertraline, and venlafaxine.

For example, the method can involve assaying a urine sample from the subject for 2, 3, 4, 5, 6, 7, 8, 9, 10, 11, 12, 13, 14, 15, 16, 17, 18, 19, 20, 25, 30, 35, 40, 45, 50, 60, 70, 80, or more of the plurality of drugs or drug metabolites listed in Table 1, Table 2, Table 3, Table 4, or Table 5.

TABLE 1
Example Polypharmacy Profile

| Drug Category | Drug | Class |
|---|---|---|
| Blood Pressure | Lisinopril | angiotensin-converting enzyme |
| | | inhibitor |
| | HCTZ | thiazide |
| | Verapamil | non-dihydropyridine calcium |
| | | channel blocker |
| | Diltiazem | non-dihydropyridine calcium |
| | | channel blocker |
| | Metoprolol | beta blocker |
| | Atenodol | |
| | Bystolic | |
| | Carvedilol | |
| | Propranolol | |
| | Amlodipine | dihydropyridine calcium |
| | Nifedipine | channel blocker |
| | Clonidine | central alpha-2 adrenergic |
| | | agonist |
| | Diovan | angiotensin 2 receptor blocker |
| | Benicar | |
| | Cozaar | |
| Cholesterol & TG | Simvastatin | statin |
| | Lovastatin | |
| | Fluvastatin | |
| | Pravastatin | |
| | Rosuvastatin | |
| | Pitavastatin | |
| | Atorvastatin | |
| | Colesevelam | bile acid sequestrant |
| | Ezetimibe | NPC1L1 protein blocker |
| Diabetic | Metformin | biguanide |
| | Glipizide | sulfonylurea |
| | Glyburide | |
| | Glimepiride | |
| | Sitagliptin | DPP 4 inhibitor |
| | Linagliptin | |
| | Saxagliptin | |
| | Alogliptin | |
| | Pioglitazone | Thiazolidinedione |
| | Insulin | Insulin |
| | Dapagliflozin | SGLT2 Inhibitors |
| | Canagliflozin | |
| Psychiatric & Related | Donezepin | acetylcholinesterase inhibitor |
| Meds | Klonopin | benzodiazepine |
| | Risperdal | atypical antipsychotic |
| | Olanzapine | |
| | Carbamazepine | mood stabilizer, carboxamide, |
| | | anti-epileptic |
| | Levetriacetam | anti-epileptic agent |
| | Suboxone | partial opioid agonist, analgesic |

TABLE 2
Example Polypharmacy Profile

| Drug Category | Drug | Class |
|---|---|---|
| Blood Pressure | Lisinopril | angiotensin-converting enzyme |
| | | inhibitor |
| | HCTZ | thiazide |
| | Verapamil | non-dihydropyridine calcium |
| | | channel blocker |
| | Diltiazem | non-dihydropyridine calcium |
| | | channel blocker |
| | Metoprolol | beta blocker |
| | Atenodol | |
| | Bystolic | |
| | Carvedilol | |
| | Propranolol | |
| | Amlodipine | dihydropyridine calcium |
| | Nifedipine | channel blocker |
| | Clonidine | central alpha-2 adrenergic |
| | | agonist |
| | Diovan | angiotensin 2 receptor blocker |
| | Benicar | |
| | Cozaar | |
| Cholesterol & | Simvastatin | statin |
| TG | Lovastatin | |
| | Fluvastatin | |
| | Pravastatin | |
| | Rosuvastatin | |
| | Pitavastatin | |
| | Atorvastatin | |
| | Colesevelam | bile acid sequestrant |
| | Ezetimibe | NPC1L1 protein blocker |
| Diabetic | Metformin | biguanide |
| | Glipizide | sulfonylurea |
| | Glyburide | |
| | Glimepiride | |
| | Sitagliptin | DPP 4 inhibitor |
| | Linagliptin | |
| | Saxagliptin | |
| | Alogliptin | |
| | Pioglitazone | Thiazolidinedione |
| | Insulin | Insulin |
| | Dapagliflozin | SGLT2 Inhibitors |
| | Canagliflozin | |
| Psychiatric & | Donezepin | acetylcholinesterase inhibitor |
| Related | Klonopin | benzodiazepine |
| Meds | Risperdal | atypical antipsychotic |
| | Olanzapine | |
| | Carbamazepine | mood stabilizer, carboxamide, |
| | | anti-epileptic |
| | Levetriacetam | anti-epileptic agent |
| | Suboxone | partial opioid agonist, analgesic |
| OTC Pain Meds | Aspirin, Goody's, BC | non-steroidal anti-inflammatory |
| | Motrin | drug, analgesic |
| | Naproxen, Aleve | non-steroidal anti-inflammatory |
| | | drug |
| | Sudafed | decongestant, alpha-adrenergic |
| | | agonist |
| | Tylenol | analgesic |

TABLE 3
Example Polypharmacy Profile

| Drug Category | Drug | Class |
|---|---|---|
| Blood Pressure | Lisinopril | angiotensin-converting enzyme |
| | | inhibitor |
| | HCTZ | thiazide |
| | Verapamil | non-dihydropyridine calcium |
| | | channel blocker |
| | Diltiazem | non-dihydropyridine calcium |
| | | channel blocker |
| | Metoprolol | beta blocker |
| | Atenodol | |
| | Bystolic | |
| | Carvedilol | |
| | Propranolol | |
| | Amlodipine | dihydropyridine calcium |
| | Nifedipine | channel blocker |
| | Clonidine | central alpha-2 adrenergic |
| | | agonist |
| | Diovan | angiotensin 2 receptor blocker |
| | Benicar | |
| | Cozaar | |
| Cholesterol & TG | Simvastatin | statin |
| | Lovastatin | |
| | Fluvastatin | |
| | Pravastatin | |
| | Rosuvastatin | |
| | Pitavastatin | |
| | Atorvastatin | |
| | Colesevelam | bile acid sequestrant |
| | Ezetimibe | NPC1L1 protein blocker |
| Diabetic | Metformin | biguanide |
| | Glipizide | sulfonylurea |
| | Glyburide | |
| | Glimepiride | |
| | Sitagliptin | DPP 4 inhibitor |
| | Linagliptin | |
| | Saxagliptin | |
| | Alogliptin | |
| | Pioglitazone | Thiazolidinedione |
| | Insulin | Insulin |
| | Dapagliflozin | SGLT2 Inhibitors |
| | Canagliflozin | |
| Psychiatric & | Donezepin | acetylcholinesterase inhibitor |
| Related | Klonopin | benzodiazepine |
| Meds | Risperdal | atypical antipsychotic |
| | Olanzapine | |
| | Carbamazepine | mood stabilizer, carboxamide, |
| | | anti-epileptic |
| | Levetriacetam | anti-epileptic agent |
| | Suboxone | partial opioid agonist, analgesic |
| Over the Counter | Aspirin, Goody's, BC | non-steroidal anti-inflammatory |
| (OTC) Pain Meds | Motrin | drug, analgesic |
| | Naproxen, Aleve | non-steroidal anti-inflammatory |
| | | drug |
| | Sudafed | decongestant, alpha-adrenergic |
| | | agonist |
| | Tylenol | analgesic |
| OTC Herbals/ | Ginkgo | |
| Supplements | St. John Wart | |
| | Niacin | |
| | Red Yeast Rice | |
| Sleep Aids | Eszopiclone | non-benzodiazepine hypnotic |
| | Zaleplon | |
| | Zolpidem | |
| | Trazadone | serotonin reuptake inhibitor |
| | Lorazepam | benzodiazepine, anxiolytic |

TABLE 4
Example Polypharmacy Profile

| Drug Category | Drug | Class |
|---|---|---|
| ADD & ADHD | Methylphenidate | Central Nervous System |
| | | Stimulants |
| | Amphetamine/ | amphetamine |
| | Dextroamphetamine- | |
| | Atomoxetine | Norepinephrine reuptake |
| | | inhibitor |
| Anxiety Meds | Alprazolam | Benzodiazepine |
| | Diazepam | Benzodiazepine |
| | Lorazepam | Benzodiazepine |
| | Buspirone | serotonin type 1A receptors |
| | | agonist |
| Blood Pressure | Lisinopril | angiotensin-converting enzyme |
| | | inhibitor |
| | HCTZ | thiazide |
| | Verapamil | non-dihydropyridine calcium |
| | | channel blocker |
| | Diltiazem | non-dihydropyridine calcium |
| | | channel blocker |
| | Metoprolol | beta blocker |
| | Atenodol | |
| | Propranolol | |
| | Amlodipine | dihydropyridine calcium |
| | | channel blocker |
| | Clonidine | central alpha-2 adrenergic |
| | | agonist |
| Cardiac | Warfarin | Vitamin K antagonist |
| | Digoxin | cardiac glycoside |
| | Clopidogrel | p2y12 platelet inhibitor |
| Cholesterol & TG | Simvastatin | statin |
| | Lovastatin | |
| | Atorvastatin | |
| | Niacin | nicotinic acid derivative |
| Chronic Pain & | Gabapentin | anti-epileptic agent |
| Fibromyalgia | Pregabalin | |
| Meds | Diclofenac | non-steroidal anti-inflammatory |
| | Indomethacin | drug |
| | Lortab, Vicodin, | narcotic pain reliever |
| | Narco, Lorcet | |
| | Darvon | |
| | Tylenol #3 | analgesic |
| | Ultram, Ultracet | opioid |
| | Percocet | |
| | Oxycontin | |
| | Morphine | |
| Diabetic & | Metformin | biguanide |
| Metabolic | Levothyroxine | thyroxine derivative |
| Meds | | |
| Migraine Meds | Sumatriptan | serotonin-1b and serotonin-1d |
| | Almotriptan | receptor agonist |
| | Zolmitriptan | |
| | Topiramate | anti-epileptic agent |
| Muscle Relaxants | Cyclobenzaprine | muscle relaxant |
| | Carisoprodol | |
| | Baclofen | gamma-aminobutyric acid-ergic |
| | | agonist |
| OTC Pain Meds | Aspirin, Goody's, BC | non-steroidal anti-inflammatory |
| | Motrin | drug, analgesic |
| | Naproxen, Aleve | |
| | Sudafed | decongestant, alpha-adrenergic |
| | | agonist |
| | Tylenol | analgesic |
| OTC Herbals/ | Ginkgo | |
| Supplements | St. John Wart | |
| | Niacin | |
| | Red Yeast Rice | |
| Other Recreational | Cotinine | |
| | Marijuana | |
| | Alcohol | |
| Psychiatric & | Donezepin | acetylcholinesterase inhibitor |
| Related | Klonopin | benzodiazepine |
| Meds | Risperdal | atypical antipsychotic |
| | Olanzapine | |
| | Carbamazepine | mood stabilizer, carboxamide, |
| | | anti-epileptic |
| | Levetriacetam | anti-epileptic agent |
| | Suboxone | partial opioid agonist, analgesic |
| Sleep | Eszopiclone | non-benzodiazepine hypnotic |
| | Zaleplon | |
| | Zolpidem | |
| | Trazadone | serotonin reuptake inhibitor |
| | Lorazepam | benzodiazepine, anxiolytic |
| Weight Loss | Phentermine | sympathomimetic amine |
| | | anorectic |

TABLE 5
Example Polypharmacy Profile

| Drug Category | Drug | Class |
|---|---|---|
| ADD & ADHD | Methylphenidate | Central Nervous System |
| | | Stimulants |
| | Amphetamine/ | amphetamine |
| | Dextroamphetamine | |
| | Atomoxetine | Norepinephrine reuptake |
| | | inhibitor |
| Anxiety Meds | Alprazolam | Benzodiazepine |
| | Diazepam | Benzodiazepine |
| | Lorazepam | Benzodiazepine |
| | Buspirone | serotonin type 1A receptors |
| | | agonist |
| Blood Pressure | Lisinopril | angiotensin-converting enzyme |
| | | inhibitor |
| | HCTZ | thiazide |
| | Verapamil | non-dihydropyridine calcium |
| | | channel blocker |
| | Diltiazem | non-dihydropyridine calcium |
| | | channel blocker |
| | Metoprolol | beta blocker |
| | Atenodol | |
| | Propranolol | |
| | Bystolic | |
| | Amlodipine | dihydropyridine calcium |
| | | channel blocker |
| | Clonidine | central alpha-2 adrenergic |
| | | agonist |
| | Diovan | angiotensin 2 receptor blocker |
| | Benicar | |
| | Cozaar | |
| Cardiac | Warfarin | Vitamin K antagonist |
| | Digoxin | cardiac glycoside |
| | Clopidogrel | p2y12 platelet inhibitor |
| Cholesterol & TG | Simvastatin | statin |
| | Lovastatin | |
| | Atorvastatin | |
| | Niacin | nicotinic acid derivative |
| Chronic Pain & | Gabapentin | anti-epileptic agent |
| Fibromyalgia | Pregabalin | |
| Meds | Diclofenac | non-steroidal anti-inflammatory |
| | Indomethacin | drug |
| | Lortab, Vicodin, | narcotic pain reliever |
| | Narco, Lorcet | |
| | Darvon | |
| | Tylenol #3 | analgesic |
| | Ultram, Ultracet | opioid |
| | Percocet | |
| | Oxycontin | |
| | Morphine | |
| Diabetic & | Metformin | biguanide |
| Metabolic | Glipizide | sulfonylurea |
| Meds | Glyburide | |
| | Glimepiride | |
| | Levothyroxine | thyroxine derivative |
| Migraine Meds | Sumatriptan | serotonin-1b and serotonin-1d |
| | Almotriptan | receptor agonist |
| | Zolmitriptan | |
| | Topiramate | anti-epileptic agent |
| Muscle Relaxants | Cyclobenzaprine | muscle relaxant |
| | Carisoprodol | |
| | Baclofen | gamma-aminobutyric acid-ergic |
| | | agonist |
| OTC Pain Meds | Aspirin, Goody's, BC | non-steroidal anti-inflammatory |
| | Motrin | drug, analgesic |
| | Naproxen, Aleve | |
| | Sudafed | decongestant, alpha-adrenergic |
| | | agonist |
| | Tylenol | analgesic |
| OTC Herbals/ | Ginkgo | |
| Supplements | St. John Wart | |
| | Niacin | |
| | Red Yeast Rice | |
| Other Recreational | Cotinine | |
| | Marijuana | |
| | Alcohol | |
| Psychiatric & | Donezepin | acetylcholinesterase inhibitor |
| Related | Klonopin | benzodiazepine |
| Meds | Risperdal | atypical antipsychotic |
| | Olanzapine | |
| | Carbamazepine | mood stabilizer, carboxamide, |
| | | anti-epileptic |
| | Levetriacetam | anti-epileptic agent |
| | Suboxone | partial opioid agonist, analgesic |
| Sleep | Eszopiclone | non-benzodiazepine hypnotic |
| | Zaleplon | |
| | Zolpidem | |
| | Trazadone | serotonin reuptake inhibitor |
| | Lorazepam | benzodiazepine, anxiolytic |
| Weight Loss | Phentermine | sympathomimetic amine |
| | | anorectic |
| GERD and Reflux | omeprazole (Prilosec) | proton-pump inhibitor |
| | lansoprazole (Prevacid) | |
| | pantoprazole (Protonix) | |
| | esomeprazole (Nexium) | |
| Erectile | Viagra (Sildenafil) | pde5 inhibitor |
| Disfunction | Cialis (Tadalafil) | |
| | Levitra (Vardenafil) | |
| Osteoporosis | alendronate (Fosamax) | bisphosphonate |
| | ibandronate (Boniva) | |
| | estrogen therapy | estrogen |

The disclosed methods can further involve detecting potential adverse drug reactions in the polypharmacy of the subject. In addition, the method can further involve selecting a drug for treating the subject that does not interact with any drugs in the polypharmacy of the subject.

Drugs or drug metabolites can be detected in a bodily fluid sample obtained from a subject using any suitable method known in the art. The bodily fluid can be any bodily fluid obtained from the subject that can be assayed to detect levels of a given drug or drug metabolite. For example, the bodily fluid can be blood, serum, plasma, urine, or combinations thereof. In certain embodiments, the bodily fluid is a urine sample obtained from the subject.

Suitable methods for detecting and/or quantifying the levels of drugs or drug metabolites in bodily fluid samples are known in the art. Appropriate methods can be selected in view of a number of factors, including the nature of the bodily fluid sample, the drugs or drug metabolites of interest, and other practical considerations.

For example, mass spectrometry (MS) can be used to determine whether or not a sample (e.g., a urine sample) contains a drug or drug metabolite. If desired, as known in the art, internal and/or external standards (e.g., isotopically-labeled internal standards and/or an external standards) can be used to quantify the levels of drugs or drug metabolites in a sample. Suitable methods of analyzing samples using MS are known in the art, and can involve, by way of example, liquid chromatography-mass spectrometry (LC/MS), gas chromatography-mass spectrometry (GC/MS), or tandem MS methods such as liquid chromatography-tandem mass spectrometry (LC-MS/MS) or gas chromatography mass spectrometry (GC-MS/MS).

A number of embodiments of the invention have been described. Nevertheless, it will be understood that various modifications may be made without departing from the spirit and scope of the invention. Accordingly, other embodiments are within the scope of the following claims.

What is claimed is:

1. A method for detecting polypharmacy in a subject, comprising assaying a urine sample from the subject for a plurality of drugs or drug metabolites from two or more drug classes selected from the group consisting of antihypertensive drugs, anti-diabetic drugs, cholesterol drugs, and psychiatric drugs.

2. The method of claim 1, wherein the antihypertensive drugs comprise at least two drugs selected from the group consisting of an ACE inhibitor, angiotensin 2 receptor antagonist, alpha-2 adrenergic receptor agonist, dihydropyridine calcium channel blocker, non-dihydropyridine calcium channel blocker, beta blocker, and thiazide diuretic.

3. The method of claim 2, wherein the antihypertensive drugs comprise at least two drugs selected from the group consisting of amlodipine, atenolol, clonidine, diltiazem, hydrochlorothiazide, lisinopril, metoprolol, propranolol, verapamil, nifedipine, nebivolol, carvedilol, valsartan, olmesartan, and losartan.

4. The method of claim 1, wherein the anti-diabetic drugs comprise at least two drugs selected from the group consisting of a biguanide, sulfonylurea, DPP 4 inhibitor, thiazolidinedione, insulin, and SGLT2 inhibitor.

5. The method of claim 4, wherein the anti-diabetic drugs comprise at least two drugs selected from the group consisting of metformin, glipizide, glyburide, glimepiride, sitagliptin, linagliptin, saxagliptin, alogliptin, pioglitazone, insulin, dapagliflozin, and canagliflozin.

6. The method of claim 1, wherein the cholesterol drugs comprise at least two drugs selected from the group consisting of statins, bile acid sequestrants, and NPC1L1 protein blockers.

7. The method of claim 6, wherein the cholesterol drugs comprise at least two drugs selected from the group consisting of atorvastatin, lovastatin, fluvastatin, pravastatin, rosuvastatin, pitavastatin, simvastatin, colesevelam, and ezetimibe.

8. The method of claim 1, wherein the psychiatric drugs comprise at least two drugs selected from the group consisting of benzodiazepine, antipsychotic drugs, anticonvulsant drugs, acetylcholinesterase inhibitor drugs, anti-epileptic agents, and partial opioid agonists.

9. The method of claim 8, wherein the psychiatric drugs comprise at least two drugs selected from the group consisting of donezepin, klonopin, diazepam, olanzapine, risperidone, carbamazepine, levetiracetam, suboxone, alprazolam, clonazepam, diazepam, lorazepam, and donepezil.

10. The method of claim 1, further comprising assaying the urine sample from the subject for drugs or drug metabolites from one or more drug classes selected from the group consisting of non-steroidal anti-inflammatory drugs (NSAIDS), acetaminophen, and decongestants.

11. The method of claim 10, wherein the NSAIDS are selected from the group consisting of acetaminophen, ibuprofen, naproxen, and salicylic acid.

12. The method of claim 11, wherein the one or more NSAID drugs are further selected from the group consisting of diclofenac and indomethacin.

13. The method of claim 10, wherein the decongestant comprises pseudoephedrine.

14. The method of claim 1, further comprising assaying the urine sample from the subject for herbal supplements or metabolites thereof.

15. The method of claim 14, wherein the herbal supplements are selected from the group consisting of ginkgo, niacin, red yeast rice, and St. John's Wart.

16. The method of claim 1, further comprising assaying the urine sample from the subject for drugs or drug metabolites from one or more drug classes selected from the group consisting of nonbenzodiazepine hypnotic drugs, anticoagulant drugs, cardiac glycosides, thyroid hormone drugs, triptan drugs, appetite suppressants, phsychostimulant drugs, azapirones, opioid drugs, muscle relaxant drugs, recreational drugs, and antidepressant drugs.

17. The method of claim 16, wherein the hypnotic drug or sleep aid is selected from the group consisting of eszopiclone, trazodone, zaleplon, and zolpidem.

18. The method of claim 16, wherein the anticoagulant drug is selected from the group consisting of clopidogrel and warfarin.

19. The method of claim 16, wherein the cardiac glycoside comprises digoxin.

20. The method of claim 16, wherein the thyroid hormone drug comprises levothyroxine.

21. The method of claim 16, wherein the triptan drug is a triptan selected from the group consisting of almotriptan, sumatriptan, zolmitriptan.

22. The method of claim 16, wherein the appetite suppressant drug comprises phentermine.

23. The method of claim 16, wherein the phsychostimulant drugs is selected from the group consisting of amphetamine, methylphenidate, and atomoxetine.

24. The method of claim 16, wherein the azapirone comprises buspirone.

25. The method of claim 16, wherein the opioid drug is selected from the group consisting of buprenorphine, codeine, morphine, oxycodone, hydrocodone, propoxyphene, and tramadol.

26. The method of claim 16, wherein the muscle relaxant drug is selected from the group consisting of baclofen, carisoprodol, and cyclobenzaprine.

27. The method of claim 16, wherein recreational drugs are selected from the group consisting of cotinine (nicotine), ethyl glucuronide or ethyl sulfate (alcohol metabolites), and tetrahydrocannabinol (THC).

28. The method of claim 16, wherein the antidepressant drug is selected from the group consisting of amitriptyline, aripiprazole, bupropion, citalopram, duloxetine, escitalopram, fluoxetine, paroxetine, sertraline, and venlafaxine.

29. The method of claim 1, further comprising detecting potential adverse drug reactions in the polypharmacy of the subject.

30. The method of claim 1, further comprising selecting a drug for treating the subject that does not interact with any drugs in the polypharmacy of the subject.
